(19) United States
(12) Patent Application Publication
(10) Pub. No.: US 2006/0034913 A1
(43) Pub. Date: Feb. 16, 2006

(54) Multiplex drug delivery device

(76) Inventors: James Gaede, Palm Springs, CA (US); Richard Albano, Moreno Valley, CA (US)

Correspondence Address: GORDON & REES LLP, 101 WEST BROADWAY, SUITE 1600, SAN DIEGO, CA, 92101, US

(21) Appl. No.: 10/918,189

(22) Filed: Aug. 13, 2004

(51) Int. Cl. A61K 9/20

(52) U.S. Cl. 424/464

(57) ABSTRACT

A capsule or shell for oral administration at the onset of an acute adverse health event (e.g. chest pain). The shell has a plurality of impermeable enclosures in which dosage units are situated. The shell and impermeable enclosures, are made from materials which promptly disintegrate in the buccal, oral, lingual mucosa. The dosage unit may also be formulated for prompt disintegration when contacted by the mucosa.

### BACKGROUND OF THE INVENTION

1. Field of the Invention

The invention is directed toward pharmaceutical formulations for treatment of urgent adverse health conditions, methods of preparing and using the formulations.

This invention relates to a delivery device and a method of delivering drug agents to the body, more particularly the invention relates to a multiplexed delivery device for delivering a combination of drug agents.

2. Description of Related Art

A problem with traditional pharmaceutical therapy for managing and treating the onset and early course of acute, urgent/emergent medical events, such as acute chest pain, stroke, or asthma attack involves haphazard administration of one or a series of medications. The medications are not delivered in a timely, reliable, consistent or therapeutic pattern.

Hundreds of thousands of deaths per year result from a variety of urgent medical illnesses, as well as millions of people per year suffering morbidity, which diminishes a subject's quality of life and productivity as a manifestation of their illness profiles.

Most patients do not receive significant or aggressive therapy early in the course of the onset of an adverse event, and many do not receive any medications at all. The sophisticated application of known treatment regimens does not occur. The application, instead, evolves slowly over the time of treatment/hospitalization, with wide-spread institutional variation, and currently the standard of care is to ascertain that the patient is discharged from the hospital on appropriate medications after the course of acute events to take at home. There is, in fact, widespread variance in consistency of these discharge recommendations from region to region and hospital to hospital. It would be a marked improvement in the standard of care to instruct the ‘at-risk patient’ in appropriate intervention in the event of life-threatening illness and have available appropriate medication combinations for timely self-administration in the occurrence of illness onset.

There has been no integrated solution to the problem, and the current state of clinical practice is such that recognized national guidelines for the management of a large number of emergent medical illness's are not adhered to in a reliable, consistent, or appropriate manner. A national and international literature review of data would clearly suggest a demonstrable need for the provision of a more useful, more readily applicable, and more consistent approach to the early management of emergent illness, such as coronary syndromes, stroke, asthma, anaphylaxis, and multiple other urgent situations.

The sooner appropriate, therapeutic medications are delivered, the more significant the reduction in severity of disease process, and the better the response to definitive in-hospital therapy. Additionally, this system will allow for an increased likelihood of the patient arriving at the hospital alive based on earlier administration of helpful medications in the disease event.

Approximately 800,000 persons per year in the United States experience acute myocardial infarction, and about 213,000 of them die. At least one half of these persons die within one hour of the onset of symptoms and before either reaching the hospital emergency room or before the arrival of emergency assistance. The earlier these useful medications are delivered, the more likely a patient is to survive long enough to reach the hospital. Generally patients arriving at the hospital have a much greater likelihood of survival having been administered medications for mitigating adverse physiological consequences of emergency events. Those having taken these medications before presentation at a hospital will have a greater likelihood of improved and more beneficial response to the definitive therapies available at the hospital. (Braunwald, E., et al., ACC/AHA 2002 Guideline Update for the Management of Patients with Unstable Angina and Non-ST Segment Elevation Myocardial Infarction, A Report of the American College of Cardiology/American Heart Association Task Force on Practice Guidelines (Committee on the Management of Patients with Unstable Angina, 2002)); “Healthy People 2010: Focus Area 12—Heart Disease and Stroke, Centers for Disease Control and Prevention; National Institutes of Health, 2004 (www.cdc.gov/cvh/hp2010/objectives.htm)); American Heart Association, Heart Disease and Stroke Statistics—2004 Update, Dallas, Tex.: American Heart Association.

To address these problems, the present invention provides a drug delivery device which is portable, carried on the subject, and self-administered at the onset or in the course of an adverse health event. For urgent adverse health events, the invention provides a device and method for simultaneous self-administration at the onset of symptoms of all the required medications, rather than an inconsistent, haphazard, sequence of medications administered over time that may not have as effective a clinical therapeutic response. Repetition of drug management protocols (i.e. the same pharmaceutical program/approach) is a major aspect of management that yields good outcomes.

The invention provides a prepackaged (containerized) portable delivery system containing medications in various combinations to optimize outcomes for patients who happen to suffer an urgent, adverse health event. The containerized device is carried on the person for immediate access in the event of an urgent situation, hence providing an earlier introduction of appropriate medications than is now presently administered by professionals. There is presently no such integrated drug delivery system available for this kind of problems.

### DISCLOSURE OF THE INVENTION

The invention is directed to a capsule or shell for oral administration. The shell includes a plurality of impermeable enclosures. In most embodiments, within each enclosure is a dosage unit of a different active drug. The drug agents are appropriately selected for treating a range of acute, adverse health event, including cardiopulmonary emergencies, emergencies involving the nervous system, endocrine and metabolic emergencies, abdominal emergencies, hypothermia, bleeding disorders, oncologic emergencies, infectious disease emergencies, anaphylaxis, poisoning emergencies.

An embodiment of the dosage unit is a tablet. The materials of the impermeable enclosures, as well as the shell, promptly dissolve or disintegrate in the lingual, buccal, glossal mucosa.

In one aspect of the invention for treating the onset of chest pain, the shell is formulated with drug agents formulated as dosage units which promptly disintegrate when contacted by the buccal, glossal, and lingual mucosa. The drug agents include aspirin, as one dosage unit in combination with other dosage units selected from one or more of statins, trinitroglycerin, antiplatelet agents, beta blockers, angiotensin converting enzyme inhibitors, angiotensin receptor blockers, beta blockers, and calcium channel antagonists.

An embodiment of the shell involves a housing which contains the shell. The housing is sized for the user to conveniently carry it, for example, in a pocket attached to a key ring. The housing has a cap or lid and a bottom, the cap easily removed by the user.

Another aspect of the invention involves a method of treating a subject for an urgent adverse health event. The method involves the step of administering at the onset or in the course of said adverse health event the shell, which contains a plurality of dosage units, each disposed in an impermeable enclosure within the shell.

The above-discussed and many other features and attendant advantages of the present invention will become better understood by reference to the following detailed description of the invention taken in conjunction with the accompanying drawings.

### BRIEF DESCRIPTION OF THE DRAWINGS

FIG. 1 is a longitudinal section of the primary dosage form.

FIG. 2 is a view of a step in the assembly of the primary dosage form.

FIG. 3 is a diagram of a process for assembling a primary dosage form.

FIG. 4 is a view of a housing.

FIG. 5 shows a housing, its components, and a shell in various stages of combination.

### MODES OF CARRYING OUT OF THE INVENTION

General Description and Definitions

The practice of the present invention will employ, unless otherwise indicated, conventional techniques within the skill of the art in (1) pharmacology, including compounding pharmacology; (2) pharmacological therapeutics; (3) physiology; (4) toxicology; (8) microbiology, (9) internal medicine and diagnostics. Such techniques are explained fully in the literature. See, e.g. Goodman & Gilman's The Pharmacological Basis of Therapeutics, eds. Joel G. Hardman, Lee E. Limbird, Tenth Edition, 2001, McGraw Hill; Basic & Clinical Pharmacology, Bernard G. Katzung, Eighth Edition, 2001, McGraw Hill; Pharmaceutical Dosage Forms and Drug Delivery Systems, Howard C. Ansel, Loyd V. Allen, Jr., Nicholas G. Popovich, Seventh Edition, 1999, Lippincott, William & Wilkins; Harrison's Principles of Internal Medicine by Eugene Braunwald M.D. (Editor), Anthony S. Fauci M.D. (Editor), Dennis L. Kasper M.D. (Editor), Stephen L. Hauser M.D. (Editor), Dan L. Longo M.D. (Editor), J. Larry Jameson M.D. (Editor). Such techniques are explained fully in the literature.

### Definitions

The following terminology will be used in accordance with the definitions set out below in describing the present invention. The terminology of these biological or medical properties, as used herein, is consistent with their use in standard medical dictionaries (e.g. Dorland's Medical Dictionary), and treatises (e.g. The Pharmacological Basis of Therapeutics, eds. Joel G. Hardman, Lee E. Limbird, Tenth Edition, 2001, McGraw Hill; Basic & Clinical Pharmacology, Bernard G. Katzung, Eighth Edition, 2001, McGraw Hill; Pharmaceutical Dosage Forms and Drug Delivery Systems, Howard C. Ansel, Loyd V. Allen, Jr., Nicholas G. Popovich, Seventh Edition, 1999, Lippincott, William & Wilkins.)

In this specification and the appended claims, the singular forms “a,” “an”, and “the” include plural referents unless the context clearly dictates otherwise. Thus, for example, reference to “an active agent” or “a pharmacologically active agent” includes a single active agent as well as two or more different active agents in combination, reference to “a carrier” includes mixtures of two or more carriers as well as a single carrier, and the like.

The terms “active agent,” “pharmacologically active agent,” “drug agent” or “drug” and “medication” are used interchangeably herein to refer to a chemical compound that induces a desired pharmacological, physiological effect. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent” and “drug” are used, then, or when an active agent such as an HMG CoA reductase inhibitor or an ACE inhibitor is specifically identified, it is to be understood that applicants intend to include the active agent per se as well as pharmaceutically acceptable, pharmacologically active salts, esters, amides, prodrugs, metabolites, analogs, etc. Active agents include inorganic and organic drugs that act on peripheral nerves, adrenergic receptors, cholinergic receptors, nervous system, skeletal muscles, cardiovascular system, smooth muscles, blood circulatory system, synoptic sites, neuroeffector junctional sites, endocrine and hormone systems, immunological system, reproductive system, skeletal system, autacoid system, tissues, organs, alimentary and excretory systems, inhibitory systems, histamine systems, body passageways, and the like.

A “dosage unit”(See FIG. 1) refers to one or more active agents formulated into a solid dosage form such as a tablet. 15. A dosage unit positioned within impermeable membranes 20 in the shell or intended to be so positioned may be referred to as a secondary dosage unit.

The terms “cholesterol-lowering agent” and “cholesterol-lowering drug” as used herein refer to a pharmacologically active, pharmaceutically acceptable agent that when administered to a human subject who has hypercholesterolemia or a dyslipidemia or a dyslipoproteinemia, has the effect of beneficially modifying total cholesterol (Total-C), LDL-C, VLDL-C, non HDL-C, and apolipoprotein B-100 (apo-B), as well as variable decreases in Trigylcerides (TG) and IDL-C and variable increases/decreases in HDL-C and apolipoprotein A-1 (apo-A). There is a favorable inhibition of LDL-C/IDL-C/VLDL-C oxidation and a stabilization effect on vascular endothelial cell function and vascular inflammatory acute phase reactants.

The term “inhibitor of the renin-angiotensin system” as used herein refers to a pharmacologically active, pharmaceutically acceptable agent that inhibits, directly or indirectly, the adverse effects of angiotensin, particularly angiotensin II. Included, without limitation, are agents that: inhibit angiotensin II synthesis; inhibit angiotensin II binding to the AT.sub.1, receptor; or inhibit renin activity.

By “pharmaceutically acceptable,” such as in the recitation of a “pharmaceutically acceptable carrier,” or a “pharmaceutically acceptable acid addition salt,” is meant herein as material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”), as in a “pharmacologically active” derivative or metabolite, refers to a derivative or metabolite having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well, e.g., therapeutically effective to reduce elevated cardiovascular risk.

“Carriers” or “vehicles” as used herein refer to conventional pharmaceutically acceptable carrier materials suitable for drug administration, and include any such materials known in the art that are nontoxic and do not interact with other components of a pharmaceutical composition or drug delivery system in a deleterious manner.

The term “controlled release” is intended to refer to any drug-containing formulation in which release of the drug is not immediate, i.e., with a “controlled release” formulation, oral administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). As discussed therein, immediate and nonimmediate release can be defined kinetically by reference to the following equation:

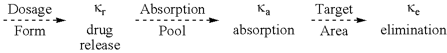

The “absorption pool” represents a solution of the drug administered at a particular absorption site, [and kᵣ, kₐ and kₑ] are first-order rate constants for (1) release of the drug from the formulation, (2) absorption, and (3) elimination, respectively. For immediate release dosage forms, the rate constant for drug release, kᵣ, is far greater than the absorption rate constant kₐ. For the controlled release formulations, i.e., for the formulations of the present invention, the opposite is true, i.e., [kᵣ<<kₐ], such that the rate of release of drug from the dosage form is the rate-limiting step in the delivery of the drug to the target area. The term “controlled release” as used herein is intended to include any nonimmediate release formulation, including but not limited to sustained release, delayed release and pulsatile release formulations. See U.S. Pat. No. 6,669,995, incorporated by reference).

The term “sustained release” is used in its conventional sense to refer to a drug formulation that provides for gradual release of drug over an extended period of time, and that preferably, although not necessarily, results in substantially constant blood levels of drug over an extended time period.

The term “delayed release” is used in its conventional sense to refer to a drug formulation in which there is a time delay provided between oral administration of a drug dosage form and the release of the drug therefrom. “Delayed release” may or may not involve gradual release of drug over an extended period of time, and thus may or may not be “sustained release.” The preferred “controlled release” formulations herein are “delayed release,” and particularly preferred “delayed release” formulations are enterically coated compositions. A form of delayed release is chronotropic release, which is designed to optimize release of drug agent(s) over a 24 hour period.

“Enteric coating” or “enterically coated” as used herein relates to the presence of polymeric materials in a drug formulation that result in an increase in the drug's resistance to disintegration in the stomach. Typically, the polymeric material is present as a coating surrounding a drug-containing core, but the polymeric material may also be present in admixture with the drug itself within a coated formulation.

By an “effective” amount or a “therapeutically effective amount” of a drug or pharmacologically active agent is meant a nontoxic but sufficient amount of the drug or agent to provide the desired effect. In the multiplex therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention or amelioration of a particular disorder or adverse physiological event in a susceptible individual as well as treatment of a clinically symptomatic individual.

The term “elevated cardiovascular risk” as used herein refers to an increased risk of incurring a cardiovascular event, peripheral vascular disease, coronary heart disease, restenosis, or atherosclerosis in an individual, such risk being due to disorders, diseases, genetic factors, behaviors, diets, or other conditions or factors. The conditions or factors that lead to elevated cardiovascular risk include, without limitation, current or prior cigarette smoking, diabetes, hypertension, hypercholesterolemia, obesity, atherosclerosis, manifest coronary artery disease, myocardial infarction, history of peripheral vascular disease, history of transient ischemic attacks or stroke, angina, systemic lupus erythematosus, hemodialysis, receiving an organ transplant, kidney disease, Chlamydia infection, Bartonella infection, and obstructive pulmonary disease.

The term “cardiovascular event” as used herein refers to a disorder or disease of the cardiovascular system having a rather sudden onset; it can also refer to a rather sudden worsening of such a disorder or disease. Examples of cardiovascular events include, without limitation: cardiac arrest, myocardial infarction, ischemia, stroke, worsening of angina, and congestive heart failure. Included in “cardiovascular events” are coronary and/or cerebrovascular event(s) and disease including myocardial infarction, myocardial ischemia, angina pectoris (including unstable angina), congestive heart failure, sudden cardiac death, cerebral infarction, cerebral thrombosis, cerebral ischemia, transient ischemic attack and the like.

The term “cerebrovascular disease” as employed herein refers to diseases including atherosclerosis of the intracranial and/or extracranial arteries, cerebral infarction, cerebral thrombosis, cerebral ischemia, stroke, and/or transient ischemic attacks.

The word “capsule” as used herein denotes its art-accepted meaning of a wall, lamina, or a membrane enclosing a drug formulation. The capsule 10 is typically a hollow shell of generally cylindrical shape having a diameter and length sufficient so that the secondary dosage forms fit appropriately in the empty capsule (See FIG. 1).

The term “multiplex” means relating to, having, or consisting of multiple elements or parts; relating to or being a system of simultaneous delivery of two or more drugs in the same dosage form; having, or consisting of multiple elements, components, or parts combined for the delivery of medicaments; relating to or being a system for the simultaneous presentation of two or more drugs in the same dosage delivery system or dosage form. A dosage form or structure preferred in the invention is a multiplex capsule 10. The multiplex capsule comprises a plurality of impermeable membrane enclosures 25. One or more pressed tablets 15 comprising drug agents are distributed in the enclosures 25. The multiplex capsule is a drug delivery device.

A dosage form preferred in the invention is a multiplexed shell or capsule, referred to herein as a primary dosage form 10, having enclosures 25.

“To multiplex”, as used herein, means to deliver a plurality of drugs or medications in simultaneous fashion using a “multiplexed” dosage form 10 or delivery device “Multiplex dosing” means the delivery of a plurality of drugs or medications formulated as dosage units and disposed within a multiplex capsule or shell prepared specifically for simultaneous use or ingestion by a patient with a disease process or disease event.

“n-part” shell means a shell for separately housing and impermeably sealing at least two dosage units from each other. An example of an n-part shell is a multiplex capsule. “n” refers to the number of enclosures, i.e. parts in the shell

Primary Dosage Form Delivery of Secondary Dosage Forms

It will be appreciated by those versed in the dispensing art, that the multiplexed capsule of the invention is a drug delivery system which comprises a primary delivery member or primary dosage form, i.e. the shell, that dispenses a plurality of secondary dosage units or secondary dosage forms into the sublingual area.

Accordingly, a primary dosage form comprises a plurality of dosage units 15 that are within secondary dosage forms 40 that are released by the primary dosage form in a preselected region of the gastrointestinal tract, such as the sublingual mucosa. In operation, the invention provides a primary delivery system for releasing a plurality of dosage units, e.g. tablets from secondary dosage forms in the sublingual area that diffuse through the sublingual mucosa and systemically disperse a delivered drug.

In the primary dosage form of the invention, active agents are formulated into dosage units, which are then enclosed in or positioned in secondary dosage forms which are impermeable enclosures within the shell or gel capsule.

Depending on the context in which it is used herein, the term “secondary dosage form” refers to an empty impermeable enclosure or an impermeable enclosure that contains a dosage unit (25, 40). In one aspect, a dosage unit positioned in an impermeable enclosure is referred to herein as a secondary dosage form. Among the dosage units used in the invention, at least one of the plurality of them is formulated wholly or partially for immediate release sublingually. The remaining secondary dosage forms are independently selected from (a) a dosage unit wholly formulated for immediate release in the sublingual area, (b) a dosage unit which is formulated of a mixture of immediate release drug composition and modified release drug composition, (c) a dosage unit wholly formulated for modified release.

Knowledge of pharmaceutical compositions and dosage forms is available to those of skill in the art for making and using the dosage forms f the invention.

### Pharmaceutical Compositions and Dosage Forms

Each particular pharmaceutical product which contains a drug is a formulation or a dosage unit 15 unique unto itself. In addition to the active therapeutic ingredients, a pharmaceutical formulation also contains a number of nontherapeutic or pharmaceutic ingredients. It is through their use that a formulation achieves its unique composition and characteristic physical appearance. Pharmaceutic ingredients include such materials as fillers, thickeners, solvents, suspending agents, anti-oxidants, emulsifiers, tablet coating and disintegrants, stabilizing agents, antimicrobial preservatives, flavors, colorants and sweeteners.

The formulation must be such that all components are physically and chemically compatible, including the active therapeutic agents, the pharmaceutic ingredients and the packaging materials.

To prepare a drug substance into a dosage form or pharmaceutical composition, pharmaceutic ingredients, which the art also refers to as adjuvants or carriers, are required. In the preparation of tablets, diluents or fillers are commonly added to increase the bulk of the formulation, binders to cause the adhesion of the powdered drug and pharmaceutic substances, anti-adherents or lubricants to assist the smooth tableting process, disintegrating agents to promote tablet break-up after administration, and coatings to improve stability, control disintegration, or to enhance appearance. For each dosage form, the pharmaceutic ingredients establish the primary features of the product, and contribute to the physical form, texture, stability, taste and overall appearance.

The principal categories of pharmaceutic ingredients are numerous and well known to those skilled in the art. Catalogs and categories and examples pharmaceutic ingredients and examples within each are found in Pharmaceutical Dosage Forms and Drug Delivery Systems, Howard C. Ansel, Loyd V. Allen, Jr., Nicholas G. Popovich, Seventh Edition, 1999, Lippincott, William & Wilkins, which is hereby incorporated by reference. In particular, attention is focused on Chapter 3—Dosage Form Design: Pharmaceutic and Formulation Considerations. Table 3.3 in this reference provides non-limiting examples of pharmaceutic ingredients, and examples thereof. It is understood that formulations of the invention include pharmaceutic ingredients and/or excipients.

The Handbook of Pharmaceutical Excipients presents monographs on over 200 excipients used in pharmaceutical dosage form preparation. Included in each monograph is such information as: nonproprietary, chemical, and commercial names; empirical and chemical formulas and molecular weight; pharmaceutic specifications and chemical and physical properties; incompatibles and interactions with other excipients and drug substances; regulatory status; and applications in pharmaceutic formulation or technology.

When two or more active agents are combined in a single pharmaceutical dosage form or unit, possible interactions among the active agents, and among the active agents and the excipients, must be considered. Such consideration is well within the purview of those skilled in the art of pharmaceutical formulation. For example, aspirin is acidic and may react with basic compounds or alkali esters in such a way as to cause hydrolysis of the aspirin and/or degradation of the other compounds. Aspirin may, for example, react with acid labile compounds such as pravastatin to degrade them.

The primary dosage forms 10 of the invention involve dosage units 15 of pharmaceutical compositions wherein active agents and/or chemically incompatible active agents are formulated into separate dosage units and separated from each other by impermeable membranes 20 within the primary dosage form.

Among dosage units which find utility in the present invention are those disclosed in U.S. Pat. Nos. 6,669,955, 6,569,457, 6,235,311, hereby incorporated by reference.

At least one of the secondary dosage units is formulated wholly or in part for instant release in the sublingual space.

It will also be appreciated by those in the art that such dosage forms, wherein two or more active agents are physically separated from the other active agents, can be manufactured so that different active agents will have different release profiles, e.g., if one active agent is formulated with an enteric coating, another active agent is formulated in a sustained release matrix, and the like. Alternatively, non-reactive pharmaceutically active derivatives of one or more of the potentially interacting compounds may be used, such as using a neutral salicylate instead of aspirin.

Secondary dosage units useful in the invention include pharmaceutical dosage forms that contain two or more multiple dosage units that are physically segregated from each other, wherein the various dosage units may have different release profiles. For example, one or more dosage units may provide immediate release of an active agent (e.g., within about an hour following oral ingestion), one or more dosage units may provide sustained release of an active agent (such that the active agent therein is gradually released over an extended time period), and one or more dosage units may provide delayed release of an active agent, wherein release following the initial “delay” may or may not be sustained release. Drug release may be made “pulsatile” in that two or more drug doses are released at spaced apart intervals of time.

### The Shell—Multiplex Gel Capsule Dosage Forms

A preferred shell or primary dosage form of the invention is a multiplexed gel capsule 10 formed from material formulated to immediately or instantly dissolve under the tongue or in the oral cavity, the desired site of delivery. The primary dosage form is formulated so that the shell 30, 35 and impermeable enclosures 25 disintegrate upon exposure of the shell and secondary dosage 15 units to the epithelial and sublingual and/or buccal mucosal tissue and the enzymes associated with those tissues.

Referring to the drawings of the multiplex primary dosage form 10 of the invention, the wall 45 surrounds and forms internal lumen 50 in the embodiment illustrated in FIG. 1. The lumen 50 comprises a multiplicity of secondary dosage forms 40 within which are secondary dosage units 15. The secondary dosage units in a preferred embodiments are pressed tablets which are symmetrically shaped, uniform diametered, and biconcave. Between secondary dosage units 15, an impermeable membrane 20 is positioned, biased against the internal wall 45 of the lumen 50, thereby forming an impermeable enclosure 25 around each secondary dosage unit 15. In certain embodiments, the shell, impermeable membranes, and secondary dosage units disintegrate immediately as they are contacted by mucous membranes and/or saliva in the buccal or sublingual environment.

The multiplex gel capsule dosage form comprises secondary dosage forms which comprise active-agent-containing compositions in solid form, including particulates such as granules, beads, powders, pellets, tablets.

So long as it immediately disintegrates in the buccal, lingual, or sublingual mucosa, suitable capsules 30, 35 may be either hard or soft, and are generally made of gelatin, starch, or a cellulosic material, with gelatin capsules preferred. Two-piece hard gelatin capsules are may be sealed, such as with gelatin bands or the like. See, for example, Remington: The Science and Practice of Pharmacy, and Pharmaceutical Dosage Forms and Drug Delivery Systems, Howard C. Ansel, Loyd V. Allen, Jr., Nicholas G. Popovich, Seventh Edition, 1999, Lippincott, William & Wilkins, which describe materials and methods for preparing encapsulated pharmaceuticals.

In accordance with the practice of this invention, capsules are made of tasteless materials that are filled easily, and they can have a variety of sizes from triple zero to five. The capsules used for the purpose of this invention can be transparent, colorless, or colored capsules can be used to give a special product a distinctive appearance. Secondary dosage units 15 and impermeable membranes 20, are inserted manually or by machine into a capsule. In place in the lumen 50, the margins of the impermeable membrane are biased against the internal wall of the lumen 50 between secondary dosage units. The capsule can be made from capsule forming materials comprising hydrophilic polymer, e.g. gelatin. Hydrophilic capsular material suitable for the primary dosage form of the invention include gelatin, starch casein, chitosan, soya bean protein, safflower protein, alginates, gellan gum, carrageenan, xanthan gum, phthalated gelatin, succinated gelatin, cellulosephtalate-acetate, polyvinylacetate, hydroxypropyl methylcellulose, polycinylacetate-phthalate, polymerisates of acrylic or methacrylic esters and mixtures thereof.

After oral administration of the primary dosage form to the sublingual area, the multiplex capsule and impermeable enclosures dissolve, dispersing to the mucosa the secondary dosage units, which immediately dissolve, dispersing all their useful drugs immediately to the mucosa for absorbtion into the systemic circulation. In the sublingual environment, the capsule wall 55 immediately begins to hydrate, loses its integrity, and releases the secondary dosage units by virtue of the materials which form the gel shell and the impermeable membranes of the enclosures. The secondary dosage forms promptly releasing their contents. The presently preferred materials of the shell are pH-sensitive, nontoxic, physiologically inactive, and do not adversely effect the drug and a host. The materials dissolve, disintegrate, degrade, hydrolyze, solubilize, are digested, or undergo like change in this biological pH environment. The product produced, as the material changes and releases the secondary dosage forms is nontoxic, chemically inert, and physiologically inactive. One group of presently preferred materials are polymers, such as proteins having a peptide bond like gelatin of the soft or hard type. In one aspect, the primary and secondary dosage forms of the invention are dose-dumping dosage forms. The therapeutic consequence of this dose-dumping is a presentation to a mucosa (e.g. glossal, lingual, buccal, or gastrointestinal) consisting of an initially high therapeutic dose of drug.

### Preparation of Multiplexed Primary Dosage Forms

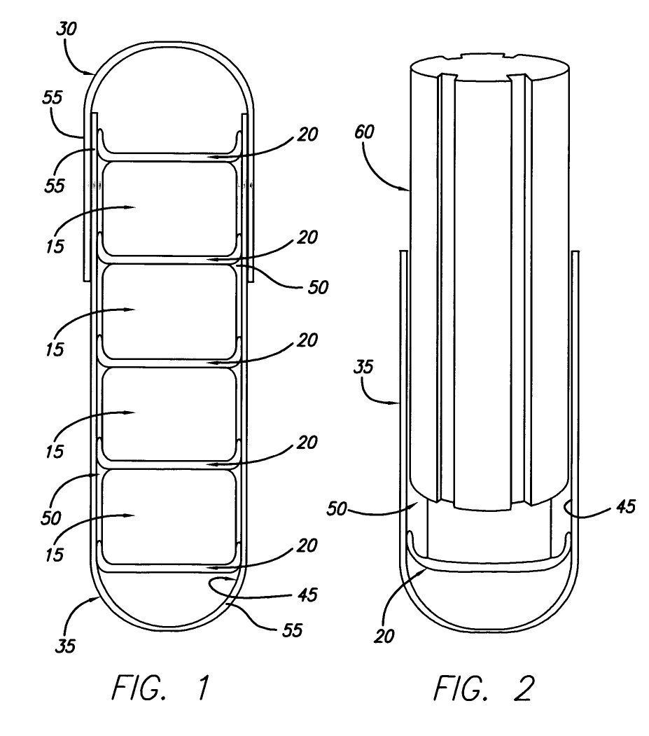

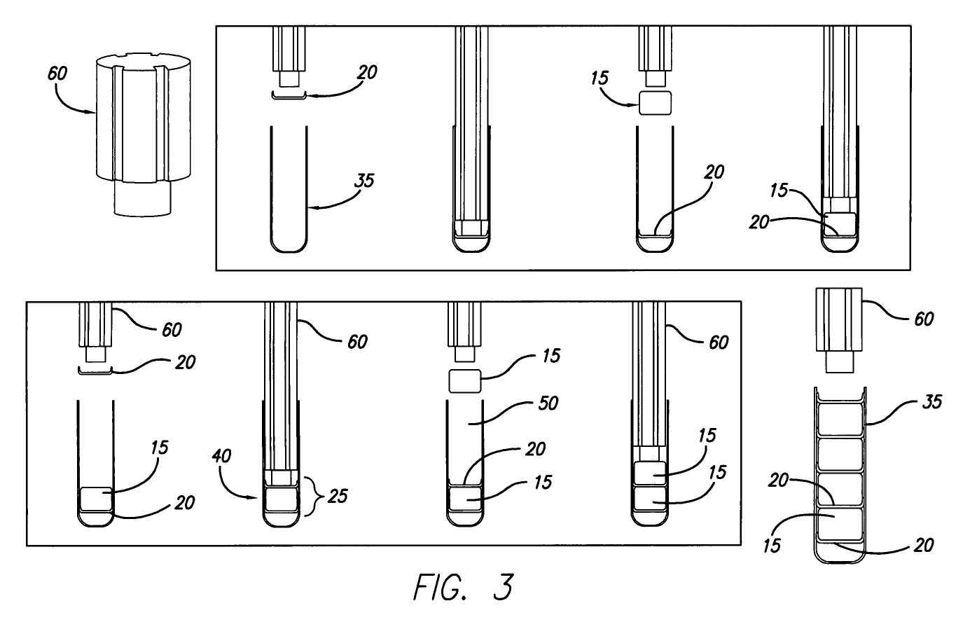

Referring to FIGS. 2 and 3, the preparation of multiplexed primary dosage forms comprising secondary dosage forms involves forming secondary dosage forms in the multiplexed primary dosage form. In most instances the amount of a particular drug placed in a primary dosage form represents a single dose of the medication.

Incompatibility of Drugs in the Presence of Each Other

Certain drugs cannot be combined due to chemical incompatibility instability in the presence of each other. This chemical incompatibility can be eliminated by the novel dosage form provided by this invention.

The present invention is a delivery system which, by virtue of its multiplex structure comprising multiple enclosures, impermeably separates secondary dosage units from each other. The multiplex shell houses dosage forms separately, and separately (albeit simultaneously in certain embodiments) dispenses them free of chemical interactions attributed to chemical incompatibility.

The impermeable enclosures 25 which comprise the multiplex shell allow for incompatible active agents to be separated until such times as they are released or, indeed, to keep active agents completely separated, delivering said agents to distinct sites in the gastrointestinal tract.

When so packaged in a multiplex capsule, the active agents are safely separated from one another during storage, i.e. as long as the impermeable enclosures are intact.

The present invention achieves dosage forms of drugs that have different rates of hydrolysis, different rates of oxidation, different rates of decomposition, different rates of delivery and different rates of bio-need into a primary dosage form that dispenses essentially free of one drug's affecting another drug during storage in the multiplex shell.

### Filling Capsule Shells

The primary dosage form multiplex capsule can be formed manually or by machine with dosage units distributed in secondary dosage forms. Secondary dosage units and impermeable membranes are alternately forced or fitted into the lumen 50 of the bottom of the gel shell. The invention includes a method for formulating the primary dosage forms of the invention, comprising the following steps.

A non-limiting method of making a primary dosage form is illustrated in FIGS. 2 and 3. A push rod 60, having a diameter sized for mating insertion of the capsule's lumen 50, plunges a secondary dosage unit through a sheet of impermeable membrane material 20 held in place by a dye at the opening of the capsule's lumen 50. The impermeable material is cut by the dye into a circular shape with a diameter from about 50% greater than the gel capsule's inner diameter. The elastic modulus and tensile strength of the impermeable material is such that, when inserted into a gel capsule bottom, the circumferential margin of the impermeable material deforms and remains biased or compressed into sealed laminate engagement with the interior wall of the lumen 50. Pushed through the impermeable membrane material, the secondary dosage unit is advanced by the rod into the capsule, i.e. inserted into the capsule, enclosed on one side by impermeable material. The next secondary dosage unit is advanced through a sheet of impermeable membrane, and into the capsule. Accordingly, the first inserted secondary dosage unit is, in effect, positioned in an impermeable enclosure formed by an impermeable membrane positioned on each end of the dosage unit, in cooperation with the side walls of the gel capsule (FIG. 1). The circumferential margin of the impermeable membrane material is tensioned in curved configuration against the capsular walls, effectively forming a seal by laminating itself with sufficient tension against the interior wall of the capsule. The curved configuration of the circumferential margin tensioned against the inner walls of the capsule functions as a shock absorber to mitigate the effects of vibration. The impermeable enclosure also mitigates against the effects of, heat, water, oxygen, and other physical traumas on the integrity of the secondary dosage units.

The primary dosage form is thus filled with secondary dosage forms, each comprising a secondary dosage unit. A secondary dosage unit is impermeably enclosed by impermeable membranes and inner side walls of the gel capsule. The capsule is then closed with the shell cap 30.

### Formulation Fundamentals: Dosage Forms

Disintegration Of Primary And Secondary Dosage Forms: For the medicinal agent in a tablet to become fully available for absorption, the tablet must first disintegrate and discharge the drug to the body fluids for dissolution. Immediate disintegration of secondary dosage units 15, e.g. tablets, is important for those medicinal agents intended to be absorbed in the mucosa where it disintegrates, e.g. lingual, glossal, buccal, or gastrointestinal. Upon tablet disintegration, the aggregate surface area of drug particles for contacting mucosa is greater than the tablet's, thereby increasing the rate at which the dose of active agent will penetrate or absorb in the mucosa faster.

### Tablets

Buccal or sublingual tablets are flat, oval tablets intended to be dissolved in the buccal pouch (buccal tablets) or beneath the tongue (sublingual tablets) for absorption through the oral mucosa. They enable the oral absorption of drugs that are destroyed by the gastric juice and/or are poorly absorbed from the gastrointestinal tract. In embodiments of the invention, tablets are designed to promptly lose their integrity for rapid mucosal absorption. Those for sublingual use (e.g. nitroglycerin sublingual tablets) dissolve promptly and provide rapid drug effects. The tablets of the invention are intended to be rapidly disintegrated and are formulated for rapid systemic absorbtion through the sublingual/buccal mucosa in the oral cavity. Methods for formulating active agents into immediate release tablets are well known in the art, as are methods for testing dissolution characteristics of solid dosage forms (Pharmaceutical Dosage Forms and Drug Delivery Systems, Howard C. Ansel, Loyd V. Allen, Jr., Nicholas G. Popovich, Seventh Edition, 1999, Lippincott, William & Wilkins, page 207).

### Enclosures

Secondary dosage forms 40 are enclosures 25 formed from impermeable membrane materials 20 which enclose or coat one or more drugs or active agents such that the drug or active agent is otherwise bound as a dosage unit within the impermeable material. In one aspect, the impermeable material is a formed from a sheet of polymeric synthetic material, biocompatible or pharmaceutically acceptable, and impermeable to molecules of the drug agents which the material encloses in the primary dosage form of the invention. The impermeable material protects the dosage unit which it encloses from environmental entry of atmospheric gases including moisture. Methods for making polymers suitable as the impermeable material are well known in the art (Polymer Biomaterials in Solution, as Interfaces and as Solids, ISBN 90-6764, 180-4, Brill Academic Publishers, Netherlands; Journal of Biomaterials Science, Polymer Edition, Brill Academic Publishers). The flexible impermeable material is sufficiently elastic and tensile so that when cut into a circular shape and inserted into a gel capsule, the inner wall of the capsule deforms the circumferential margin into a laminate biased against the inner wall.

An impermeable material which finds use in the invention for forming enclosures within the capsule involves pressed fibrous membrane. Non-limiting examples of material used for making pressed fibrous membranes include fillers (which are otherwise commonly used to add necessary bulk to a drug formulation to prepare tablets of a desired size): lactose monohydrate, microcrystalline cellulose, metharylic acid, polyethylene glycol, gylceryl monostearate, triethyl citrate, magnesium carbonate, hydroxypropyl cellulose, hypromellose, mannitol, gelatin, talc, magnesium stearate, polyacrylate polyethylene glycol. BHT or other antioxidants may be added as a preservative agent. Embodiments of the pressed fibrous membrane include flavoring or coloring agents to ameliorate undesirable tastes of the medications and/or to color code various dosage forms to avoid mistakes in assembly, prescription, dispensing or self-administration.

Methods of making pressed fibrous membranes are well known in the art of biopolymer science. One method involves a filler material combined in a water slurry to allow appropriate distribution of materials. The slurry is laid out either in sheets or individual die forming devices (to give a final shape) and either allowed to air dry (ambient desiccation), baked dry at moderate temperatures (55-60° C.) (thermal desiccation), or freeze dried (sublimation), or subject to vacuum (desiccation via alteration of vapor pressure). Each approach gives a slightly different physical characteristic such as stiffness, bendability, compressibility, and these characteristics are selected according to the physical properties desired for making particular embodiments of the multiplex dosage form.

Another methods involves placing commercially available puffed rice in a blender or similar device and reducing it to powder. Well known methods are available for converting rice or other grains into blends of naturally derived polymeric carbohydrates and protein (e.g. Rice Chemistry and Technology, 3^{rd} edition, edited by Elaine T. Champagne) suitable for forming impermeable membranes. The powder is combined into a slurry formed by the addition of water, magnesium stearate and microcrystalline cellulose and povidone along with mannitol. The slurry is placed in a flat pan or form dies and subject to any one of the above desiccant procedures.

If prepared in a flat pan, the large sheets are then separated sharply into half-inch strips, wound around a two-inch spool, and cut to shape by a variety of sharp die-type cutting instruments. In one embodiment, the shape of the membrane after die-cutting is a circle approximately 0.4 mm larger than the internal diameter of the capsular shell housing.

Another embodiment of the secondary dosage form involves film-coated tablets, which are compressed tablets coated with an impermeable layer of a polymer capable of forming a skin-like film over the tablet. By its composition, the coating is designed to rupture and expose the core tablet at the desired location buccal or sublingual location. Methods of film-coating tablets are known in the arts of making pharmaceutical dosage forms. See; Pharmaceutical Dosage Forms and Drug Delivery Systems, Howard C. Ansel, Loyd V. Allen, Jr., Nicholas G. Popovich, Seventh Edition, 1999 at pp. 221-223.

### The Housing

### Packaging and Storage of The Primary Dosage Forms

The proper packaging, labeling and storage of pharmaceutical products are essential for product stability and efficacious use.

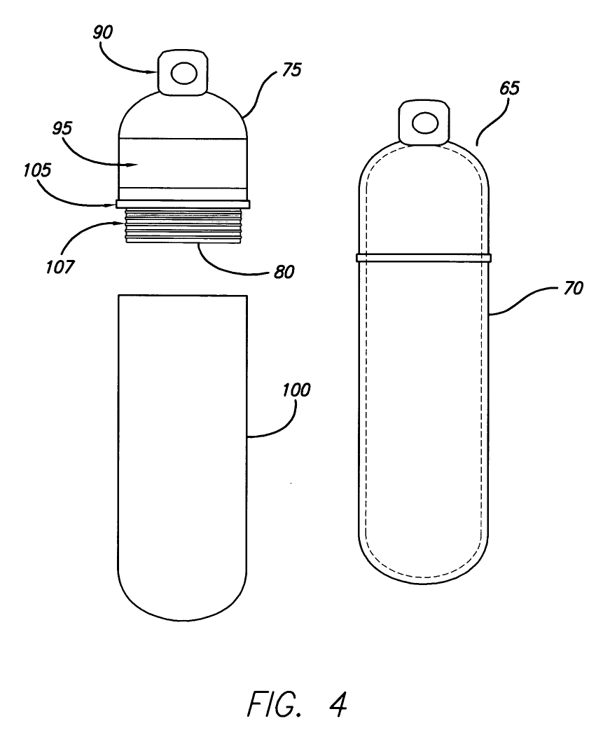

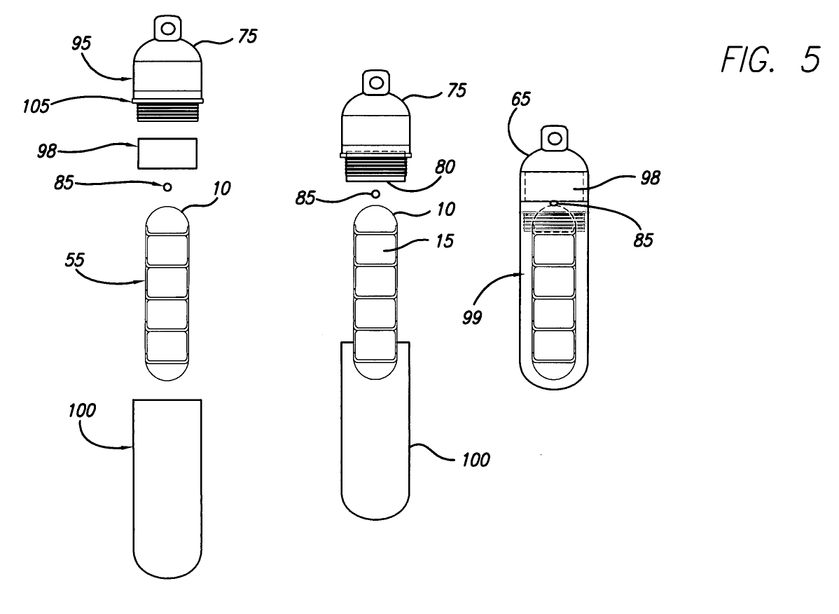

Primary dosage forms of the invention are stored preferably in removably-lidded, tight, desiccated containers (FIGS. 4 and 5). Products that are prone to decomposition by moisture generally are co-packaged with a desiccant packet. Drugs that are adversely affected by light are packaged in light-resistant containers. Primary and secondary dosage forms that are properly stored will remain stable for several years or more.

### Containers

Standards for the packaging of pharmaceuticals by manufacturers are contained in the Current Good Manufacturing Practice section of. Code of Federal Regulations in the United States Pharmacopoeia/National Formulary, and in the FDA's Guideline for Submitting Documentation for Packaging for human Drugs and Biologics

### Containers for Dispensing Primary Dosage Forms

Specifications listed in the USP prescribe the type of container suitable for the repackaging or dispensing of the primary dosage forms of the invention. Depending on the item, the container might be required to be tight, well-closed and light resistant.

A single dose container is one in which the quantity of drug contained is intended as a single dose. In one aspect of the invention, the primary dosage form is disposed or stored in single-unit or multiple-unit housing or container 70. A single-unit housing is designed to hold a quantity intended for administration as a single dose of medication(s) when opened. A single-unit housing is termed a unit-dose when dispensed to a patient. The single-unit housing or packaging of drugs may be performed on a large scale by a manufacturer or distributor or on a smaller scale by the pharmacy dispensing the medication.

Some pharmaceutical manufacturers use unit-of-use packaging; that is packaging in which the quantity of drug product prescribed is packaged in a container for dispensing. For example, if a primary dosage form capsule formulated according to the present invention is prescribed for one-time administration, unit-of-use packaging, i.e. the housing, would contain one primary dosage form capsule.

Many pharmaceutical products require light resistant containers to protect them from photochemical deterioration. In most instances a container made of a good quality of amber glass or a light resistant opaque plastic will reduce light transmission sufficiently to protect a light-sensitive pharmaceutical. The USP provides tests and standards for glass and plastic containers with respect to their ability to prevent the transmission of light. Metal materials are among the materials which are suitable for housings, although other materials, such as certain plastics and glasses, which are equally impenetrable by light, moisture, vapor or gas, especially oxygen, may be employed.

For storing or shipping, the multiplex dosage forms of the invention, prior to placement in a housing, are suitably disposed in “high-barrier” packaging to provide added protection to the pharmaceutical agents against the effects of humidity’. High barrier packaging (Dosage Forms and Drug Delivery Systems, Howard C. Ansel, Loyd V. Allen, Jr., Nicholas G. Popovich, Seventh Edition, 1999, Lippincott, William & Wilkins at p. 158) meets ‘the drug stability requirements for packaging adopted by the International Committee on Harmonization which call for the long-term testing of packaged products for a minimum for 12 months at 25° C. (±2 degrees) at 60 percent relative humidity. Desiccant protectants (e.g. silica gel in small packets) may be included as added protection against the effects of moisture vapor.

In one embodiment of the housing, the interior bottom of the housing is shaped as a saddle or seat for the primary dosage form capsule to prevent or limit movement of the capsule when carried around by the user, and prevent chafing of the capsule against the inner walls of the housing. Preferably, the primary dosage form is stored in the housing under pressured (32 psi) nitrogen 99 in the housing (FIG. 5).

An embodiment of the invention comprises a housing 65 which has a cap 75 and a bottom 100, and which contains a primary dosage form 10, which comprises secondary dosage units disposed in a plurality of impermeable enclosures as secondary dosage forms. The capsule or shell of the primary dosage form contains pressed hard tablets 15 partitioned by impermeable inter-tablet membranes 20, i.e partitioning membranes between the tablets. The housing is preferably designed to mitigate the effects of vibration, heat, water, oxygen, and other physical traumas on the integrity of the primary dosage form, and its secondary dosage form contents.

### The Cap/Lid

As used herein, the terms “engaged housing and cap,” “sealed housing and cap,” “storage housing,” “housing and cap combination” refer to the capped preservation housing in which one or more primary dosage forms are contained.

In one aspect, the housing of the present invention provides a cap 75 having an inner surface 80 on which an adhesive material 85 is disposed for releasably fixing at least one primary dosage form 10 to the cap 75. At least a portion of the shell's surface adheres to the adhesive. Upon removing the cap, the primary dosage form is presented to the user for easy retrieval and self-administration by plucking the shell from the lid with the lips, teeth, or fingers, thereby admitting the shell into the buccal, glossal or perilingual environment. This embodiment operates best for caps with large enough diameters so that the wall of the cap does not interfere with the user's administration of the primary dosage form. The present invention advantageously facilitates the extraction of a shell from the housing for those in exigent circumstances and/or with an impairment of the hands.

In another embodiment, the adhesive is disposed on the end of a stem fixed to the inside surface of a cap. The term “inside surface” here means the surface of the cap that faces the inside of a housing when the cap seals the housing, and is meant to encompass any liners or gaskets fixed to the cap that face the inside of a housing. When the housing is sealed with the cap, the stem extends from the cap into the housing. At least one shell contacts and releasably adheres to the adhesive at the end of the stem. When the cap is removed, the shell is conveniently presented to the consumer to be plucked by mouth or fingers from the end of the stem. This is especially advantageous for use with bottles with narrow mouths or necks, and for small shells.

In yet another embodiment, the adhesive is disposed on the side of a stem fixed to the inside surface of a cap, the stem extending into the bottle when the cap seals the bottle. When the cap is removed, the pill is presented to the consumer on the side of the stem. This embodiment is advantageous for a wide range of shell sizes and housing mouth and neck diameters. The housing is diametrically and length dimensioned to accommodate one or more shells.

The present invention may be configured to extract more than one shell from a housing by arranging more than one separate adhesive area on the inside of a cap or on a stem. One pill is fixed to each adhesive area, advantageously and reliably providing a multi-shell dosage to the consumer.

The present invention provides a means for reliably, safely and cleanly extracting at least one shell in a controlled fashion from a wide variety of housings by a consumer with either healthy or impaired hands. The present invention is advantageously and easily usable by those who have limited or impaired use of their hands, or are in exigent circumstances, especially when compared to previously known methods of extracting shells from a housing.

An roughly circular or semi-spherical adhesive area from about one twenty fifth to one half of the diameter of a shell releasably fixes a single shell such that the adhesive area remains fixed to the cap or stem when the consumer removes a shell releasably fixed to the adhesive area. Adhesive areas smaller than one twenty fifth the diameter of a shell are too small to reliably fix a shell, whereas adhesive areas more than one half the diameter of a shell often fix more than one shell per adhesive area. One tenth the diameter of a shell generally best suffices to fix a single shell, although adhesive areas from one twenty fifth to one half the size of the shell have been found to be effective in fixing a single shell. More than one shell may be fixed per adhesive area, but a precise number of shells is most reliably fixed if each adhesive area is dimensioned to releasably fix only one shell at a time. A suitable adhesive is FILM GRIP™ 33-4044, formerly 72-3326, manufactured by the National Starch and Chemical Company, Adhesives Division, 10 Finderne Avenue, Bridgewater, N.J. FILM GRIP™ is an adhesive formulated as a pressure sensitive emulsion adhesive, having about 59% solids, a viscosity of about 1600 cps, a pH of about 4.6, and a density of about 8.6 pounds per gallon. FILM GRIP™ is an adhesive with good wet tack that adheres well to difficult surfaces, such as polyolefins. FILM GRIP™ has very good cohesive strength, and sticks to itself sufficiently to ensure that an insignificant amount (if any) of the adhesive continues to adhere to a shell once the shell is removed from the adhesive by the consumer. (U.S. Pat. No. 5,826,747, incorporated by reference).

In one version, the cap or lid has a device 90 for attaching the housing to a key ring, neck-encircling or other limb-encircling element such as a necklace or bracelet, in any case a chain, strap or plastic or equivalent cord which is adapted to be worn about the neck of the user and which is operatively connected with a depending housing. The housing, thus hung and slidingly suspended, is convenient and readily accessible for use by the user.

In one embodiment, the inner wall of the housing at its upper end comprises a screw threaded surface with either twist or pop off threading and adapted to be closed by a readily attachable and detachable upper screw cap having a threaded surface 107 for mating with the threaded surface of the housing. It is also intended that in some embodiments, the cap or the cap-receiving section of the housing will comprise O-rings 105, washers, or other structures to facilitate sealing of the container. As used herein, the term “housing engaging means” refers to any means by which the cap of the present invention becomes attached to the housing. The lid or cap can be threaded in a variety of manners to allow a twist or pop release mode for cap removal. It is contemplated that various housing engaging means will find use in the present invention, including, but not limited to, threads.

In one embodiment, a holder 95 for moisture absorbing granules, i.e. desiccative media 98, is positioned in the cap. As used herein, the term “desiccant” refers to any material or compound that is useful for drying. Desiccants, include, but are not limited to compounds such as CaCl₂ CaO, NaOH, MgO, CaSO₄ (e.g., Drierite™), H₂ SO₄, silica gel, Mg(ClO₄)₂, and P₂O₅, commercially available from various sources, including Fisher and Multisorb. In one embodiment of the present invention waxed silica gel tablets may be used as the desiccant. However, any desiccant that is capable of providing and retaining 1-3% moisture within the vial and cap combination may be used in the present invention.

As used herein, the term “immobilized desiccant” refers to the placement of desiccant within the cap of the present invention in a manner such that the desiccant is retained within the cap.

### Method of Dispensing/Administering Primary Dosage Forms

Accordingly, the invention provides a method of dispensing a primary dosage form which comprises the steps of providing a housing 65 which contains a primary dosage form 10. The housing has a cap having an inside surface provided with an adhesive area, the primary dosage form releasably fixed to the adhesive area. The cap is removed from the housing and the primary dosage form is removed from the adhesive area in a step which involves the user administering the primary dosage form to the buccal or sublingual area.

### Acute Adverse Health Events

The primary dosage forms of the present invention are, in one aspect, formulated for treating a patient suffering from an acute adverse health event, i.e. an illness event. The primary dosage forms are preferably administered to the patient at the time of an event or immediately thereafter. The primary dosage forms of the invention, when administered in this manner, are particularly useful for increasing the likelihood or potential that a patient suffering from an acute adverse health event (a) will survive the event, and (b) will have less morbidity as an outcome of the event had the primary dosage form not been administered.

The primary dosage forms and methods of administering them are suitable for treating a wide variety of emergent medical conditions. One of skill in the art of clinical pharmacology can readily identify the combination of drug agents which those of skill in the art routinely administer to patients at the beginning of or early in the course of an acute adverse health event, as exemplified below. In the Examples section below, the specification discloses, in relation to emergent health conditions, drug combinations which are formulated as secondary dosage units within secondary dosage forms within primary dosage forms of the invention.

Emergent, acute or adverse health events or conditions are categorized as follows: (Harrison's Principles of Internal Medicine by Eugene Braunwald M.D. (Editor), Anthony S. Fauci M.D. (Editor), Dennis L. Kasper M.D. (Editor), Stephen L. Hauser M.D. (Editor), Dan L. Longo M.D. (Editor), J. Larry Jameson M.D. (Editor):

TABLE 1
Acute Adverse Health Events

Cardiopulmonary Emergencies
Chest pain
Pulmonary edema
Cyanosis and hypoxia
Shock
Cardiovascular collapse and arrest
Bradyarrhythmias
Tachyarrythmias
Cardiac tamponade
Traumatic heart disease
Acute myocardial infarction
Malignant hypertension
Aortic dissection
Asthma
Pulmonary thromboembolism
Adult respiratory distress syndrome
Drowning
Abdominal Emergencies
Abdominal pain
Acute diarrhea
Gastrointestinal bleeding
Acute intestinal obstruction
Acute appendicitis
Acute cholecystitis
Emergencies Involving the Nervous System
Syncope
Acute confusional states and coma
Tetanus
Botulism
Rabies
Viral encephalitis
Seizures
Stroke
Hypertensive encephalopathy
Spinal cord compression
Trauma of the head and spinal cord
Acute meningitis
Opiate intoxication
Central nervous system drug intoxication
Endocrine and Metabolic Emergencies
Acute renal failure
Thyroid storm and myxedema coma
Acute adrenal insufficiency
Diabetic ketoacidosis and hyperosmolar coma
Hypoglycemia
Hyper- and hypocalcemia
Other Emergencies
Hypothermia
Bleeding disorders
Oncologic emergencies
Cholera
Anaphylaxis
Electrical injuries
Heavy metal poisoning
Poisoning and its management
Gastrointestinal or diarrheal illnesses
Chemical and biological warfare

### Therapeutic Classification of Drugs

The following classification of drugs, which is non-limiting, is derived from Goodman & Gilman's The Pharmacological Basis of Therapeutics, eds. Joel G. Hardman, Lee E. Limbird, Tenth Edition, 2001, McGraw Hill, incorporated by reference for the subject matter disclosed herein. The primary dose forms of the invention comprise combinations of secondary dose forms selected from one or more of the following therapeutic categories of drug agents:

Drugs Affecting Renal and Cardiovascular Function

These include diuretics; vasopressin and other agents affecting renal conservation of water; renin and angiotensin; drugs for treating myocardial ischemia; antihypertensive agents and drugs for treating hypertension; drugs for treating heart failure; antiarrhythmic drugs; drugs for treating hypercholesterolemia

Drugs Acting at Synaptic and Neuroeffector Junctional Sites

These agents affect neurotransmission in the autonomic and somatic motor nervous systems. Included are muscarinic receptor agonists and antagonists: anticholinesterase agents; agents acting at the neuromuscular junction and autonomic ganglia; catecholamines, sympathomimetic drugs, and adrenergic receptor antagonists; 5-hydroxytryptamine (serotonin): receptor agonists and antagonists.

Drugs Acting on the Central Nervous System

These include general anesthetics; hypnotics and sedatives; drugs for treating psychiatric disorders, such as depression, anxiety disorders, psychosis, mania; drugs for treating epilepsies; drugs for treating central nervous system degenerative disorder; opioid analgesics; drugs for treating drug addiction and drug abuse.

Autacoid; Drug Therapy of Inflammation

These include histamine, bradykinin, and their antagonists; lipid derived autocoids: eicosainoids and platelet activating factor; analgesic-antipyretic and anti-inflammatory agents and drug employed in the treatment of gout; drugs used in the treatment of asthma and dyslipidemia.

Drugs Affecting Gastrointestinal Function

These include agents for control of gastric acidity and treatment of peptic ulcers and gastroesophageal reflux disease; prokinetic agents, antiemetics, and agents used in irritable bowel syndrome; agents used for diarrhea, constipation, and inflammatory bowel disease; agents used for biliary and pancreatic disease.

Chemotherapy of Parasitic Infections

These include agents used in the chemotherapy of protozoal infections, for example, malaria, amebiasis, giardiasis, trichomoniasis, trypanosomiasis, leishmaniasis; and for treating helminthiasis;

Chemotherapy of Microbial Diseases

These include antimicrobial agents such as sulfonamides, trimethoprimsulfamethoxazole, quinolones and agents for urinary tract infections; penicillins, cephalosporins, and other beta-lactam antibiotics; aminoglycosides; protein synthesis inhibitors; drugs used in chemotherapy of tuberculosis, mycobacterium avium complex disease, and leprosy. Further included are antifingal agents, antiviral agents, and antiretroviral agents.

Chemotherapy of Neoplastic Diseases

These include alkylating agents, nitrogen mustards, ethylenimines and methylmelamines; alkyl sulfonates; nitrosoureas; folic acid analogs; pyrimidine analogs; purine analogs; natural products such as vinca alkaloids, paclitaxel, epipodophyllotoxins; camptothecin analogs; antibiotics such as dactinomycin, daunorubicin, doxorubicin, idarubicin; bleomycin, mitomycin; platinum coordination complexes; hydroxyurea; porocarbazine; adrenocorticosteroids; aminoglutethimide and other aromatase inhibitors; antiestrogens (e.g. tamoxifen); gonadotropin-releasing hormone analogs; antiandrogens; biological response modifiers such as interleukins, granulocyte colony stimulating factor, granulocyte/macrophage colony-stimulating factor; monoclonal antibodies.

Drugs Used for Immunomodulation

These include immunosuppressive agents, tolerogens, and immunostimulants. These drugs include vaccines based on compositions of antibodies ranging from immune globulin to purified antibody compositions to monoclonal antibody compositions.

Drugs Acting on the Blood and the Blood-Forming Organs

These include hematopoietic agents, such as growth factors, minerals and vitamins; and anticoagulant, thrombolytic, and antiplatelet drugs.

Hormones and Hormone Antagonists

These include pituitary hormones and their hypothalamic releasing factors; thyroid and antithyroid drugs; estrogens and progestins; androgens; adrenocorticotropic hormone; adrenocortical steroids and their synthetic analogs; inhibitors of the synthesis and actions of adrenocortical hormones; insulin, oral hypoglycemic agents; agents affecting calcification and bone turnover: calcium, phosphate, parathyroid hormone, vitamin D, calcitonin.

### Methods of Managing Acute/Emergent Conditions

The invention provides a method of treating patients at the beginning or in the midst of acute emergent health conditions (see Table I). The treatment involves oral administration, preferably to the sublingual area, of a primary dosage form which comprises a combination of dosage units as secondary dosage forms. The primary dosage form shell, which is multiplexed, is formulated for prompt disintegration in the lingual, glossal, buccal areas, and dispersal of the secondary dosage units from formerly impermeable enclosures. The material of the enclosures is formulated for immediate disintegration and rapid dispersal of the component drug agents into the sublingual/buccal mucosa for rapid systemic absorption through the mucosa. The primary dosage form comprises a multiplex shell to administer two, three, four or an even higher number of secondary dosage forms comprising a plurality of drug agents.

The primary and secondary dosage forms of the delivery device invention are formulated for rapid release of a predetermined number of active drug agents to the preferred site of delivery, namely, the lingual, glass, buccal, or gastrointestinal mucosa.

Once ingested, the primary dosage form is quickly broken down and the contents made available, the user showing an increase in the blood level concentration of the active principle(s).

With an adroit choice of the dissolution profiles, i.e. the rate at which the active agent(s) within the secondary dosage forms dissolve, the primary dosage form, which is a delivery device, can be arranged to provide the desired pharmacodynamic and pharmacokinetic profiles for treatment of a particular acute adverse health event. This can also be augmented by the provision of the same active agent in a particular phase or in particular phases (immediate vs. controlled-release) within each multiplexed dosage form.

### EXAMPLES

### Example 1

### Chest Pain, Myocardial Infarction, Management of Acute Chest Pain Syndromes, Management of Acute Coronary Syndromes (ACS)

The present invention provides a drug delivery device 10, the multiplexed shell or capsule comprising secondary dosage forms, and a method for appropriate, early delivery of medications using the device for the management and treatment of acute coronary syndrome, i.e. ischemic chest pain syndromes, unstable angina, and acute myocardial infarction, to improve patient clinical outcome.

It is well known and well documented in the medical literature that early usage of aspirin, anti-platelet agents, ‘statin-type’ drugs, trinitroglycerin, and in frequent cases, ‘beta-blockers’ and ‘ACE-inhibitors’, and angiotensin receptor blockers are of importance in the treatment of coronary artery disease manifesting as ischemic chest pain. In most cases, it is the current ‘state-of-the-art’ to provide these medications in the treatment of ischemic heart disease, and the literature demonstrates that the earlier they are administered, the better the outcome for the patient with this disease process. Additionally, it has become a recognized national standard to be sure that patients are discharged from the hospital on various combinations of medications known to those of skill in the clinical arts to improve outcomes (See Improved Outcome References at end of specification).

In this non-limiting example, the invention provides primary dosage forms comprising combinations of pharmaceutical agents formulated as dosage units in secondary dosage forms for managing and treating ischemic chest pain syndromes, unstable angina, and acute myocardial infarction.

Secondary dosage forms are formulated as tablets for rapid dispersal in the lingual, glossal, buccal or gastrointestinal mucosa. The tablets (secondary dosage units) are disposed in impermeable enclosures within the multiplexed capsule.

A combination of two to seven drugs, each drug formulated individually as a tableted dose unit, is selected from pharmaceutical agents well known to those in the art for treating chest pain, myocardial infarction, management of acute chest pain syndromes.

The following list comprises drug agent categories, examples within each, and representative, non-limiting dosages of the dosage units which comprise them:
Trinitroglycerin—1/150 or a grain, or 0.4 mg
Aspirin—81 mg

Antiplatelet Agents:
Ticlopidine 125 or 250 mg
Clopidogrel 75 mg
Dipyridamole 25 mg or 50 mg

Statins:
Lovastatin 20 or 40 mg
Fluvastatin 20 or 40 mg
Simvastatin 10 or 20 mg
Pravastatin 20 or 40 mg
Atorvastatin 10 or 20 mg
Rosuvastatin 5 or mg

ACE (Angiotensin Converting Enzyme) Inhibitors:
Quinapril 5 mg
Perindopril 2 mg
Ramipril 2.5 mg
Captopril 12.5 mg
Enalapril 5.0 mg
Benazepril 5 mg
Trandolapril 1 mg
Fosinopril 10 mg
Lisinopril 5 mg
Moexipril 7.5 mg

ARB (Angiotensin Receptor Blockers)s:
Candesartan 4 mg
Irbesartan 75 mg
Olmesartan 10 or 20 mg
Losartan 25 mg
Telmisartan 20 or 40 mg
Eprosartan 300 mg

Beta Blockers (BB):
Bisoprolol 2.5 mg
Labetalol 100 mg
Metoprolol 25 mg
Atenolol 25 mg
Pindolol 5 mg
Acebutolol 200 mg
Betaxolol 2.5 mg
Carvedilol 3.125 mg
Nadolol 20 mg
Penbutolol 10 mg
Propranolol 20 mg
Timolol 5.0 mg

CCA (Calcium Channel Antagonists):
Nifedipine 10 mg
Verapimil 40 mg
Diltiazem 30 mg
Isradipine 5 mg
Felodipine 2.5 mg
Amlodipine 5.0 mg
Nisoldipine 10 mg
Nicardipine 20 mg

Embodiments of the invention are numerous and the possible combinations expressed as combinatorial selections of representative secondary dosage units disposed in secondary dosage forms in the multiplexed capsule (primary dosage form) of the invention.

For example, in a combinatorial series in which one of the tablets (dosage units) is aspirin, the other dosage units in the primary dosage form are accommodated by the invention:
1. Aspirin (81 mg) and an agent selected from the group consisting of statins, trinitroglycerin, and an antiplatelet agent.
2. Aspirin and statin, and further comprising an agent selected from the group consisting of: trinitroglycerin, antiplatelet agents, BB, ACE, ARB, CCA.
3. Aspirin and trinitroglycerin, further comprising an agent selected from the group consisting of antiplatelet agent, BB, ACE, ARB, CCA.
4. Aspirin and an antiplatelet agent, further comprising an agent selected from the group consisting of BB, ACE, ARB, CCA.
5. Aspirin, statin, trinitroglycerin, and an antiplatelet agent, said capsule further comprising an agent selected from the group consisting of BB, ACE, ARB, and CCA.
6. Aspirin, statin, trinitroglycerin, an antiplatelet agent and a BB, said capsule further comprising an agent selected from the group consisting of ACE, ARB, and CCA.
7. Aspirin, statin, trinitroglycerin, an antiplatelet agent, a BB, and an ARB, said capsule further comprising an agent selected from the group consisting of CCA and ACE.
8. Aspirin, statin, trinitroglycerin, an antiplatelet agent, ACE, and CCA, said capsule further comprising an agent selected from the group consisting of ARB and BB.
9. Aspirin, statin, trinitroglycerin, an antiplatelet agent, ACE, ARB, CCA and BB.

The following sets forth examples of active agents pressed into tablets and enclosed in impermeable enclosure materials. Preferable embodiments of enclosures are formulated with a suitable preservative, such as BHT, and/or various coloring agents such as: yellow or rideferric oxide, FD&C Blue 2, FD&C Yellow 10, silicon dioxide,
Lovastatin: cellulose, lactose, magnesium stearate, starch
Nitroglycerin tablets: lactose monohydrate, glyceryl monostearate, pregelatinized starch, calcium or magnesium stearate
Ticlid: citric acid, magnesium stearate, microcrystalline cellulose, povidone or crospovidone), starch, stearic acid
Aspirin: starch (plain or pregelatinized), cellulose (microcrystalline or not)
Beta Blockers: magnesium stearate, hypromellose, lactose, povidone, cellulose (or starch)
ACE Inhibitors: starch, magnesium stearate, calcium phosphate, mannitol
Calcium Channel Blockers: cellulose (microcrystalline or not), magnesium Stearate, starch (sodium starch glycolate), dibasic calcium phosphate anhydrous (helpful for shelf life of ccb's)
Angiotensin Receptor Blocker: stearate, cellulose (e.g. hydroxypropyl methylcellulose), Crospovidone, microcrystalline cellulose

The invention provides a multiplexed dosage form, which in various embodiments, is contained in a housing (e.g. a container), and a method for treating chest pain with a patient accessible, portable containered, series of medications proven to decrease myocardial damage and improve short/long term outcomes. Using the present invention, one of skill in the art with reference to the literature of pharmacological treatment of acute cerebrovascular events, acute asthma events, acute anaphylactic events can devise therapeutically effective or prophylactically effective dosage amounts for inclusion in primary dosage forms for early onset treatment.

### Example 2

### Acute Asthma

The present invention provides a device and a method for appropriate, early delivery of medications for the management and treatment of acute asthma events to improve patient clinical outcome.

It is well known and well documented in the medical literature that early use of the following agents are of importance in the treatment of acute asthma events. In most cases, it is the current ‘state-of-the-art’ to provide these medications in the treatment of acute asthma. The earlier they are administered, the better the outcome for the patient with this disease process. Additionally, it has become a recognized national standard to be sure that patients are discharged from the hospital on such medications to improve outcomes.

In this non-limiting example, the invention provides primary dosage forms comprising combinations of pharmaceutical agents formulated in secondary dosage forms for managing and treating acute asthma.

Secondary dosage forms are formulated as tablets for rapid dispersal in the sublingual mucosa. The tablets are disposed in impermeable enclosures with the multiplex capsule.

A combination of two to seven drugs is selected from pharmaceutical agents well known to those in the art for treating acute asthma. The following non-limiting list comprises drug agent categories, and examples within each, and representative, non-limiting dosages for adults, with the understanding that the dosages can be customized for children on a mg/kg or mg/square meter or age-related basis according to dosage regimens well known in the art.

The following doses assume an adult patient, however they can be customized for child on an mg/kg or mg/meter squared or age related basis:

Steroid—
Methylprednisolone 40 mg (0.5 mg/kg)

Leukotriene Receptor Antagonist—
Zalfirlukast 10 mg (for age 6 and up, same dose—not for under five years
Montelukast 10 mg (for age less than one—not recommend)
(for age 12-23 months—4 mg)
(for age 2-5—4 mg)
(for age 6-14—5 mg)
(for age 15 and up—10 mg)

Methylxanthine—
Theophylline 200 mg for adult patients, and 4 mg/kg for pediatric patients

Beta Agonists—
Albuterol 4 mg
evalbuterol 0.075 mg

Anti-Histamines—
Loratadine 5.0 mg
Fexofenadine 60 mg
Diphenhydramine 25 mg
Brompheniramine 12 mg
Chorpheniramine 8 mg
Carbinoxamine 4 mg
Pyrilamine 30 mg
Acrivastine 8 mg
Azatadine 1 mg
Centirizine 5 mg
Parabenzaime 25 mg
Azelastine 1 mg

Expectorants—
Guaifenesin 300 or 600 mg

Anticholinergics—
Ipratropium 75 mcg

### Example 3

### Acute Urticaria

For the early treatment of acute urticaria, appropriate active agents are employed in the device and method of the invention according to this non-limiting example:

Steroid—
Methylprednisolone—from 40 or 100 mg

H-1 Antihistamines—
Loratadine 10.0 mg
Fexofenadine 120 mg
Diphenhydramine 50 mg
Brompheniramine 24 mg
Chorpheniramine 16 mg
Carbinoxamine 8 mg
Pyrilamine 60 mg
Acrivastine 16 mg
Azatadine 2 mg
Centirizine 10 mg
Parabenzaime 50 mg
Azelastine 2 mg
(For children, maximum dosages of antihistamines are calculated on a mg/kg basis)

Leukotriene Receptor Antagonist—
Zalfirlukast 10 mg (for age 6 and up, same dose—not for under five years
Montelukast 10 mg (for age less than one—not recommend)
(for age 12-23 months—4 mg)
(for age 2-5—4 mg)
(for age 6-14—5 mg)
(for age 15 and up—10 mg)

Sympathomimetic Agents—
Pseudoephedrine 75 mg
Ephedrine 1 mg

H-2 Antihistamines—
Cimetidine 400 mg
Rantadine 300 mg
Famotidine 20 or 40 mg
Nizatidine 300 mg

### Dosing

The dose administered is adjusted according to age, weight and condition of the patient, as well as the route of administration, dosage form and regimen and the desired result.

It is apparent that it is not necessary herein to specify dosages or method of use of the primary and secondary dosage forms of the inventions as it is known to those one skilled in the art of clinical pharmacology, pharmaceutical formulation, internal medicine, and emergency medicine can readily deduce suitable unit doses for various active agents. Those of skill in the art, based on knowledge of compounds having similar physiological or biological activity, would be able to discern appropriate dosages and methods of use without undue experimentation. Goodman & Gilman's The Pharmacological Basis of Therapeutics, eds. Joel G. Hardman, Lee E. Limbird, Tenth Edition, 2001, McGraw Hill; Basic & Clinical Pharmacology, Bernard G. Katzung, Eighth Edition, 2001

Improved Outcome References

(Braunwald, E., et al., ACC/AHA 2002 Guideline Update for the Management of Patients with Unstable Angina and Non-ST Segment Elevation Myocardial Infarction, A Report of the American College of Cardiology/American Heart Association Task Force on Practice Guidelines (Committee on the Management of Patients with Unstable Angina, 2002)); “Healthy People 2010: Focus Area 12—Heart Disease and Stroke, Centers for Disease Control and Prevention; National Institutes of Health, 2004 (www.cdc.gov/cvh/hp2010/objectives.htm)); American Heart Association, Heart Disease and Stroke Statistics—2004 Update, Dallas, Tex.: American Heart Association; ASCOT Trial. Server P S, Dahlof B, Poulter N R, et al. Prevention of coronary stroke events with atorvastitin in hypertensive patients who have average or lower than average cholesterol concentrations. Lancet. 2003; 361:1149-1158; Braunwald E. the open-artery theory is alive and well-again. N Engl J. Med. 1993; 329(22):1650-1652; Zeymer U, Tebbe U, Essen R, et al. Influence of time to treatment on early infarct-related artery patency after different thrombolytic regimens. ALKK-Study Group. Am Heart J 1999; 137(1):34-38; Welsh R C, Ornato J, Armstrong P W. Prehospital management of acute ST-elevation myocardial infarction: a time for reappraisal in North America. Am Heart J 2003; 145(1):1-8; Chan A W, Moliterno D J, Berger P B, et al for the TARGET Investigators. Triple anti-platelet therapy during percutaneous coronary intervention is associated with improved outcomes including 1-year survival. Results from the Dotirofivan and Reopro give similar efficacy outcome trial. (TAGET). J Am Coll Cardiol. In Press; Dupuis J, Tardif J C, Cernacek, P, et al. Cholesterol reduction rapidly improves endothelial function after acute coronary syndromes. The RECIFE (Reduction of Cholesterol in Ischemia and Function of the Endothelium) trial. Circulation. 1999(25):3227-3233; Ridker P M, Rifai N, Pfeffer M A, et al. Inflammation, prevastatin and the risk of coronary events after myocardial infarction in patients with average cholersterol levels. Cholesterol and Recurrent Events. (CARE). Investigators. Circulation 1998; 98(9):839-844; Aronow H D, Topol E J, Roe M T, et al. Effect of lipid-lowering therapy on early mortality after acute coronary syndromes: an observational study. Lancet. 2001; 357(9262):1063-1068; Senestrand U, Wallentin L, Early statin treatment following acute myocardial infarction and 1 year survival. JAMA. 2001; 285(4):430-436; Gaspardone A, Crea F, Versaci F, et al. Predictive value of C-reactive protein after successful coronary-artery stenting in patients with stable angina. Am J Cardiol. 1998; 82(4)515-518; Versaci F, Gasparsdone A, Tomai F, et al. Predictive value of C-reactive protein in patients with unstable angina pectoris undergoing coronary artery stent implantation. Am J Cardiol 2000; 85(1):92-95, A98; Pfeffer M A, Braunwald E. Ventricular remodeling after myocardial infarction: Experimental observations and clinical implications. Circulation 1990; 81:1161-72; Cleland J G F, Erhardt L, Murray G, et al. Effect of ramipril on morbidity and mode of death among survivors of acute myocardial infarction with evidenced of heart failure. A report from the AIRE study investigators. Eur Heart J 1997; 18:41-51; Pfeffer M A, Braunwald E. Moye L A, et al. Effect of captopril on mortality and morbidity in patients with left ventricular dysfunction after myocardial infarction: results of the survival and ventricular enlargement trail. N Engl J Med 1992; 327:669-77; The Survival of Myocardial Infarction Long-term Evaluation (SMILE) Study Investigators. The effect of the angiotensin-converting-enzyme inhibitor zofenopril on mortality and morbidity after anterior myocardial infarction. N Engl J Med 1995; 332:80-5; The Norwegian Multicenter Study Group. Timolol-induced reduction in mortality and reinfarction in patients surviving acute myocardial infarction. N Engl J Med 1981; 304:801-7; Beta-Blocker Heart Attack Trial Research Group. A randomized trail of propranolol in patients with acute myocardial infarction. 1. Mortality results. JAMA 1982; 247:1707-14; Wilhelmesson C, Vedin A. Beta blockers in ischemic heart disease (Goteburg metoprolol trial) Am J Cardiol 1983; 52 (2): 108A-12; Beta-Blocker Heart Attack Trail Research Group. A randomized trial of propranolol in patients with acute myocardial infarction. I (BHAT). Mortality results. JAMA 1982; 247(12):1707-14; Sever P S. The Anglo-Scandinavian Cardiac Outcomes Trial: Morbidity-mortality outcome from lipid-lowering in a hypertensive population. ACC 2003: American College of Cardiology 52nd Annual Scientific Session; Mar. 30-Apr. 2, 2003 Chicago, Ill. Late Breaking Clinical Trials III #421-3; Dahlof B, Devereux R B, Kjeldsen S E, for the LIFE Study Group. Cardiovascular morbidity and mortality in the Losartan Intervention For Endpoint reduction in hypertension study (LIFE): a randomized trial against atenolol. Lancet. 2002; 359:995-1003. Abstract; Scandinavian Simvastatin Survival Study Group. Randomized trial of cholesterol lowering in 4444 patients with coronary heart disease: the Scandinavian Simvastatin Survival Study (4S). Lancet. 1994; 344:1383-1389; Shepard J, Cobbe S M, Ford I, et al, for the West of Scotland Coronary Prevention Study Group. Prevention of coronary heart disease with Pravastatin in men with hypercholesterolemia. N Engl J. Med. 1995; 333:1301-1307; Sacks F M, Pfeffer M A, Moye L A, et al, for the Cholesterol and Recurrent Events Trial Investigators. The effect of pravastatin on coronary events after myocardial infarction in patients with average cholesterol levels. N Engl J. Med. 1996; 355:1001-1009; Long-term Intervention with Pravastatin in Ischemic Disease (LIPID) Study Group. Prevention of cardiovascular events and death with pravastatin in patients with coronary heart disease and a broad range of initial cholesterol levels. N Engl J. Med. 1998; 339: 1349-1357; Downs J R, Clearfield M, Weis S, et al, for the AFCAPS/TexCAPS Research Group. Primary prevention of acute coronary events with lovastatin in men and women with average cholesterol levels: results of AFCAPS/TexCAPS. JAMA. 1998:279:1615-1622; Schwartz G C, Olsson A G, Ezekowitz M D, et al, for the Myocardial Ischemia Reduction with Aggressive Cholesterol Lowering (MIRACL) Study Investigators. Effects of atorvastatin on early recurrent ischemic events in acute coronary syndromes. JAMA. 2001; 285:1711-1718; Pitt B, Waters D, Brown W V, for the Atorvastatin versus Revascularization Treatment Investigators. Aggressive lipid lowering therapy compared with angioplasty in stable coronary artery disease. N Engl J. Med. 1999; 341:70-76; Heart Protection Study Collaborative Group. MRC/BHF Heart Protection Study of cholesterol lowering with simvastatin in 20,536 high risk individuals: a randomized placebo controlled trial. Lancet. 2002; 360:7-22; Antiplatelet Trailists'Collaboration. Collaborative overview of randomized trials of antiplatelet therapy: prevention of death, myocardial infarction, and stroke by prolonged antiplatelet therapy in various categories of patients. BMJ1994; 308:81-106; Steering Committee of the Physicians' Health Study Research Group. Final report on the aspirin component of the ongoing physicians' health study. N Engl J Med 1989; 321:129-35; Early Treatment Diabetic Retinopathy Study Investigators. Aspirin effects on mortality and morbidity in patients with diabetes mellitus. JAMA 1992; 268:1292-300; Antiplatelet Trialists' Collaboration. Collaborative overview of randomized trials of antiplatelet therapy: prevention of death, myocardial infarction, and stroke by prolonged antiplatelet therapy in various categories of patients. BMJ 1994; 308:81-106; Yusuf S, Wittes J, Friedman L. Overview of results of randomized clinical trials in heart disease. Treatment following myocardial infarction. JAMA. 1988; 260(14):2088-2093; Gottileb S, McCarter R, Vogel R. Effect of beta-blockade on mortality Med. 1998; 339:489-497; The MERIT-HF Study Group: Metoprolol CR/XL Randomized Intervention Trial in Congestive Heart Failure (MERIT-HF) Lancet. 1999; 353:2001-2007; Marion D J, Fazio S, Linton M F. Current perspectives on statins. Circulation. 2000; 101:207-213; Rosenson R, Tangney C. Antiatherothrombotic properties of statins. JAMA. 1998; 279:1643-1650; Bosch J, Yusuf S, Pogue J, et al., on behalf of the HOPE investigators. Use of ramipril in preventing stroke: double blind randomized trial. BMJ. 2002, 324:699-702; Perindopril Protection Against Stoke Study (PROGRESS) Lancet 2001; 358:1033-1041; Collins R, Petro R, Baigent C, et al. Aspirin, heparin, and fibrinolytic therapy in suspected acute myocardial infarction. Drug Therapy 1997; 36:847-860; Patrono C: Aspirin as an antiplatelet drug. N Engl J Med 330: 1287, 1994; Serruyus et al. Statin reduces major adverse cardiac events in at-risk patients update on the LIPS Study, JAMA, June 2002; 287:3215-3222; Haskell W L, Alderman E L, et al, Effects of intensive multiple risk factor reduction on coronary atherosclerosis and clinical cardiac events in men and women with coronary artery disease. The Stanford Coronary Risk Intervention Project. (SCRIP). Circulation, Vol 89, 975-990; Pravastatin Acute Coronary Treatment (PACT) Study; Albert M A, Danielson E, et al for the PRINCE Investigators. Effect of statin therapy on C reactive protein levels: the pravastatin inflammation/CRP evaluation. (PRINCE) JAMA 2001; 286:64-70; Fonarow G C, Gawliniski A et al. Improved treatment of coronary heart disease presentation of a cardiac hospitalization atherosclerosis management program (CHAMP) Am J Cardiol 2001; 87:819-822; Smith S C, Blair S N, Bonow R O, et al. AHA/ACC Guidelines for preventing heart attack and death in patients with atherosclerotic cardiovascular disease: 2001 Update. A statement for healthcare professionals from the American Heart Association and the American College of Cardiology. J Am Cardiol. 2001:38:1581-1583; Fung H L, Chung S J, Bauer J A, Chong S, Kowaluk E A. Biochemical mechanism of organic nitrate action. Am J Cardiol. 1992; 70(suppl 5):4B-10B; Abrams J, Hemodynamic effects of nitroglycerin and long-acting nitrates. Am Heart J. 1985; 110:216-224; Brown B G, Bolson E, Peterson R B, Pierce C D, Dodge H T, The mechanisms of nitroglycerin action:stenosis vasodilatation as a major component of the drug response. Circulation; 1981; 64:1089-1097; Needlemen P, Jakschik B, Johnson E M Jr. Sulfhydryl requirement for relaxation of vascular smooth muscle. J Pharmacol Exp Ther. 1973; 187:324-331; Bussmann W D, Passek D, Seidel W, Kaltenbach M Reduction of CK and CK-MB indexes of infarct size by intravenous nitroglycerin. Circulation. 1981; 63:615-622; Myocardial infarct size, expansion, and complications: effect of timing, dosage, and infarct Jugdutt B I, Warnica J W, Intravenous nitroglycerin therapy to limit location. Circulation. 1988; 78:906-919; ISIS-4: a randomized factorial trial assessing early oral captopril, oral mononitrate, and intravenous magnesium sulfate in 58,050 patients with suspected acute myocardial infarction. Lancet. 1995; 345:659-675; Balsano F, Rizzon P, Violi F, et al. Antiplatelet treatment with ticlopidine in unstable angina: a controlled multicenter clinical trial: The Studio della Ticlopidina nell Angina Instabile Group. Circulation 1990; 82:17-26; Rentrop K P, Blanke H, Karsch K R, et al. Acute myocardial infarction: intracoronary application of nitroglycerin and streptokinase. Clin Cardiol. 1979; 2:354-363; Deedwania P, Beta Blockers and Cardiac Arrhythmias. New York, N.Y.: Marcel Dekker Inc. 1992; Yusuf S, Peto R, Lewis J, Collins R, Sleight P Beta blockade during and after myocardial infarction: an overview of the randomized trials. Prog Cardiovasc Dis. 1985; 27:335-371; The MIAMI Trial Research Group. Metoperolol in acute myocardial infarction: patient population. Am J Cardiol. 1985; 56:1G-57G; Pfieffer M A, Hennekens C H, When a question has an answer: rationale for our early termination of the HEART Trial. Am J Cardiol. 1995; 75:1173-1175; Latini R, Maggioni A P, Flather M, Sleight P, Tognoni G. ACE-inhibitor use in patients with myocardial infarction: summary of evidence from clinical trials. Circulation 1995; 92:3132-3137; Ambrosioni E, Borghi C, Magnani B. The effect of the angiotensin-converting-enzyme inhibitor zoefenopril on mortality and morbidity after anterior myocardial infarction: The Survival of Myocardial Infarction: Long-Term Evaluation (SMILE) Study Investigators, N Engl J. Med. 1995; 332; 80-85.))

1. A shell comprising:
a. a plurality of impermeable enclosures;
b. one or more dosage units of active drug agent disposed in said enclosures.

2. The shell of claim 1 wherein said dosage units are tablets.

3. The shell of claim 1 wherein said tablets are selected from the group of dosage forms consisting of immediate-release and modified-release forms.

4. The shell of claim 1 wherein said drug agent is selected the from a group of drug agents for the treatment of an acute adverse health condition selected from the group consisting of cardiopulmonary emergencies, emergencies involving the nervous system, endocrine and metabolic emergencies, abdominal emergencies, hypothermia, bleeding disorders, oncologic emergencies, infectious disease emergencies, anaphylaxis, poisoning emergencies, chemical and biological warfare, gastrointestinal and diarrheal illnesses.

5. The shell of claim 1 wherein said acute adverse health event is chest pain and said active drug agents comprise aspirin and one or more agents selected from the group consisting of trinitroglycerin, antiplatelet agents, statins, angiotensin converting enzyme inhibitors, angiotensin receptor blockers, beta blockers, and calcium channel blockers.

6. The shell of claim 5 wherein one of the drug agents comprises aspirin and the other drug agents are selected from the group consisting of statins, trinitroglycerin, and antiplatelet agents.

7. The shell of claim 6 comprising aspirin and statin, and further comprising an agent selected from the group consisting of trinitroglycerin, antiplatelet agents, beta blockers, ACE inhibitors, ARBS, and CCBs.

8. The shell of claim 6 comprising aspirin and trinitroglycerin, and further comprising an agent selected from the group consisting of antiplatelet agents, beta-blockers, ACE inhibitors, ARBs, and CCBs.

9. The shell of claim 6 comprising aspirin and an antiplatelet agent, and further comprising an agent selected from the group consisting of beta blockers, ACE inhibitors, ARBs, and CCBs.

10. The shell of claim 5 comprising aspirin, statin, trinitroglycerin, and an antiplatelet agent, and further comprising an agent selected from the group consisting of beta blockers, ACE inhibitors, ARBs, and CCBs.

11. The shell of claim 6 comprising aspirin, statin, trinitroglycerin, an antiplatelet agent and a beta blocker, and further comprising an agent selected from the group consisting of ACE inhibitors, ARBs, and CCBs.

12. The shell of claim 6 comprising aspirin, statin, trinitroglycerin, an antiplatelet agent, a BB, and an ARB, and further comprising an agent selected from the group consisting of CCBs and ACE inhibitors.

13. The shell of claim 6 comprising aspirin, statin, trinitroglycerin, an antiplatelet agent, an ACE inhibitors, and a CCB, and further comprising an agent selected from the group consisting of ARBs and beta blockers.

14. The shell of claim 6 comprising aspirin, statin, trinitroglycerin, an antiplatelet agent, ACE inhibitors, ARB, CCB and beta blocker.

15. The shell of claim 1 wherein said impermeable membrane is formed from material selected from 1 the group consisting of lactose monohydrate, microcrystalline cellulose, metharylic acid, polyethylene glycol, gylceryl monostearate, triethyl citrate, magnesium carbonate, hydroxypropyl cellulose, hypromellose, mannitol, talc, magnesium stearate, polyacrylate and polyethylene glycol.

16. The shell of claim 1 further comprising a housing in which said shell is disposed.

17. The shell of claim 16 wherein said housing comprises a container having a lid.

18. The shell of claim 17 wherein the inner surface of said lid comprises an adhesive and said shell is releasabaly fixed to said adhesive.

19. The shell of claim 1 wherein said shell is disposed in a releasably sealed container.

20. A method of treating a subject for an urgent adverse health event comprising the step of administering at the onset or in the course of said adverse health event a shell comprising a plurality of impermeable enclosures and one or more dosage units of active drug agent disposed in said enclosures.

21. The method of claim 20 wherein said drug agents are selected from the group of drug agents for the treatment of an acute adverse health condition selected from the group consisting of cardiopulmonary emergencies, emergencies involving the nervous system, endocrine and metabolic emergencies, abdominal emergencies, hypothermia, bleeding disorders, oncologic emergencies, infectious disease emergencies, anaphylaxis, poisoning emergencies, chemical and biological warfare, gastrointestinal and diarrheal illnesses.
